(19) United States
(12) Patent Application Publication
(10) Pub. No.: US 2007/0055330 A1
(43) Pub. Date: Mar. 8, 2007

(54) Superficial heat modality for therapeutic use

(76) Inventor: Brock T. Rutherford, Bend, OR (US)

Correspondence Address: Lorri W. Cooper;JONES DAY, North Point, 901 Lakeside Avenue, Cleveland, OH, 44114, US

(21) Appl. No.: 11/517,966

(22) Filed: Sep. 8, 2006

Related U.S. Application Data
(60) Provisional application No. 60/715,296, filed on Sep. 8, 2005.

(51) Int. Cl. A61F 7/00

(52) U.S. Cl. 607/114

(57) ABSTRACT

A heat pack system includes an inductive charging unit, a heat pack, and a pack cover. The inductive charging unit has an antenna for emitting magnetic energy. The heat pack includes two layers of a heat retaining elastomeric material with an energizing layer of material sandwiched between the layers of elastomeric material. The heat pack may include an RFID tag and an RTD lead for reading the temperature of the energizing layer and communicating this information to the inductive charging unit in order to heat the heat pack inductively. The pack cover is made of a washable material and is configured to enclose the heat pack therewithin. A chemical compound for forming the elastomeric layers is also described, as is a method for manufacturing a heat pack.

### CROSS-REFERENCE TO RELATED APPLICATION

This application claims priority to U.S. Provisional Application No. 60/715,296, filed on Sep. 8, 2005, the disclosure of which is incorporated herein by reference in its entirety.

### FIELD

This application concerns a superficial heat modality for therapeutic use. In particular, the application concerns a heat pack that is used for superficial heating.

### BACKGROUND

Silica gel in a canvas pack has been used therapeutically for many years for superficial heating. The silica gel packs are heavy and require great lead time in order to bring them to the required temperature. Silica gel packs are brought to temperature by submersion in a large, hot water bath, called a hydrocollator. It is difficult to regulate the temperature of silica gel packs and burning can result. In addition, reheat time is at least 15 minutes.

### SUMMARY

A heat pack system is shown and described. In addition, a method for manufacturing a heat pack is described. A chemical compound used in forming a heat pack layer is also described.

### BRIEF DESCRIPTION OF THE DRAWINGS

FIG. 1 is a perspective view of an example heat pack and pack cover;

FIG. 2 is a perspective view of an example heat pack and pack cover applied to a body part of a subject;

FIG. 3 is a perspective view of an example heat pack and pack cover positioned on an induction charging unit for heating purposes;

FIG. 4 is a perspective view of the example heat pack and pack cover shown prior to positioning on an alternative example induction charging unit;

FIG. 5 is a cross-sectional view of one example heat pack and pack cover;

FIG. 6 is an expanded view of the various layers and components of the heat pack and pack cover of FIG. 5;

FIG. 7 is a view of an encased RFID tag with associated RTD leads for use with the example heat pack;

FIG. 8 is an expanded view of the RFID tag of FIG. 7;

FIG. 9 is a perspective view of the example heat pack and pack cover, showing a removable layer about to be coupled to the bottom of the heat pack;

FIG. 10 is a cross-sectional view of another example heat pack and pack cover; and

FIG. 11 is an exploded view of the various layers of the heat pack and cover of FIG. 10.

### DETAILED DESCRIPTION

An example heat pack system 10 utilizes an induction charging unit 12, a heat pack 14, and a pack cover 16. The example heat pack 14, as described herein, is an improvement on existing superficial heat modalities. The heat pack 14 utilizes induction heating in order to heat a heat pack to a prescribed temperature that may be selected in advance by the practitioner, resulting in greater flexibility and accuracy for treating patients of all heat tolerance levels. In addition, the heat pack 14 is easily and accurately heated or reheated in a time period of approximately 3 minutes or less, flexible for effective use on any number of body parts, thin and light weight for very easy manageability, hypoallergenic, easily washable, and easily disinfectable. All of these features results in a heat pack 14 that effectively and safely delivers heat to a subject.

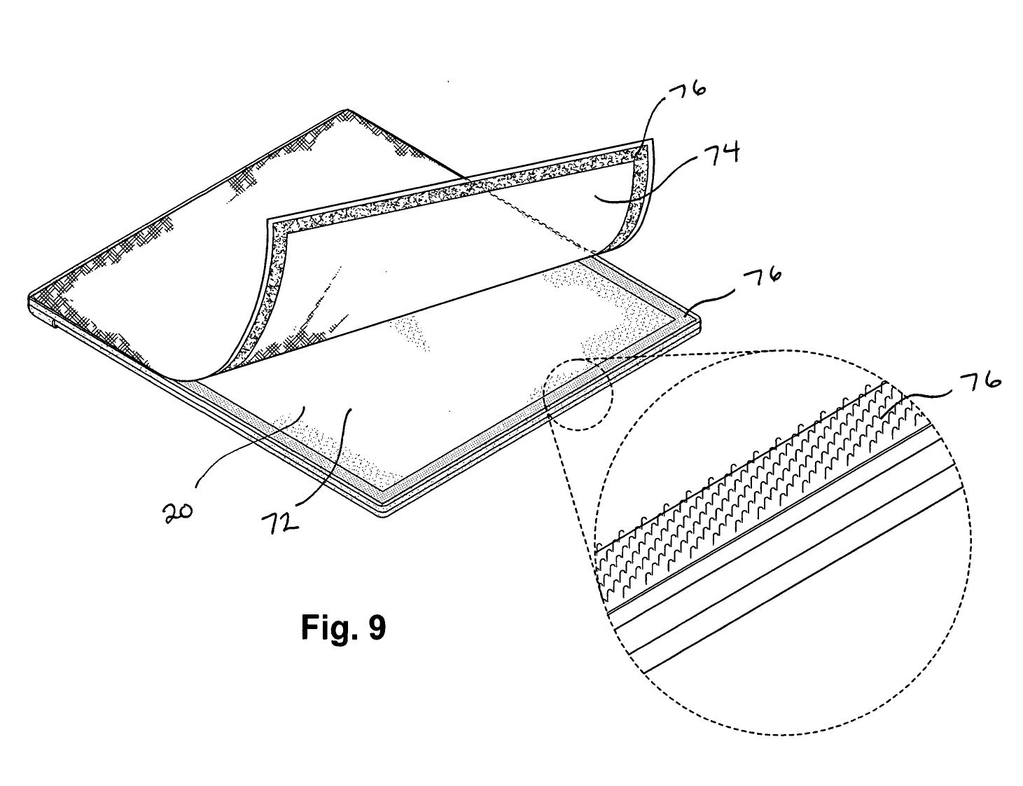

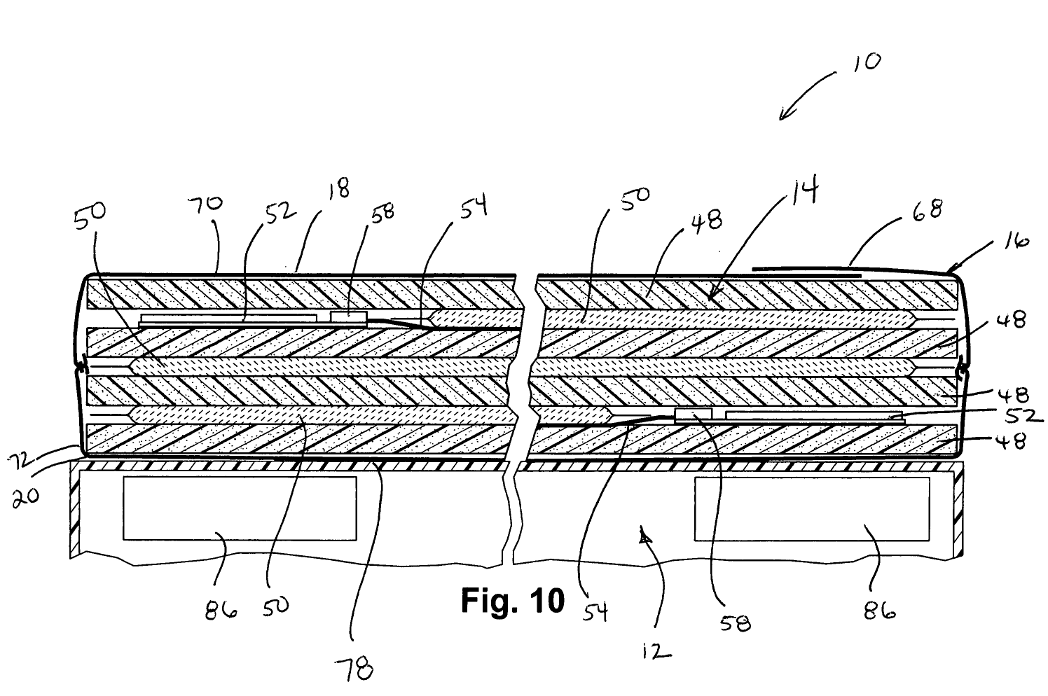

The example heat pack 14 and cover 16 are depicted in FIGS. 1-6 and 9-11. The heat pack 14 is used in conjunction with the exterior pack cover 16 and is positioned inside the pack cover 16. An example of the top 18 of the exterior pack cover 16 is shown in FIG. 1 and an example bottom layer 20 of the pack cover 18 is depicted in FIG. 9. The cover 16 shown is rectangular in shape. However, any size or shape may be utilized with the example heat pack 14. The heat pack shape can be customized to particularly difficult to heat areas of the body, for example. Round, rectangular, triangular, oval, irregular, or any other shapes may be utilized for the heat packs 14 and pack covers 16. The pack cover 16 is sized based upon the size of the heat pack 14.

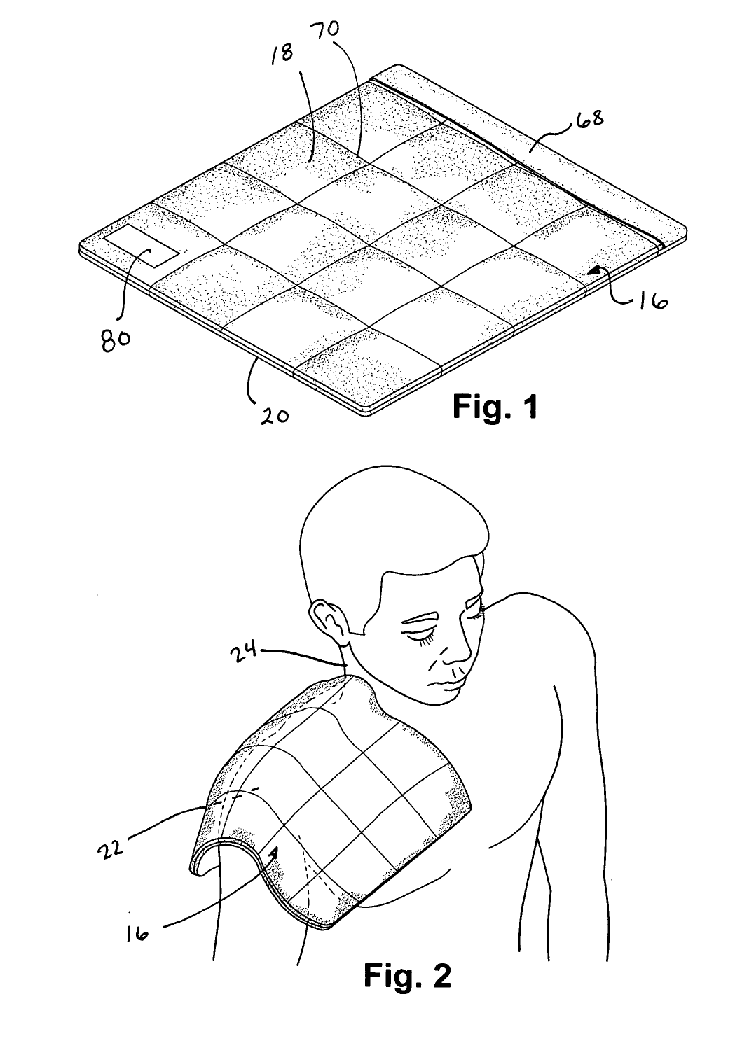

As shown in FIG. 2, the example heat pack 14 and pack cover 16 are positioned on the shoulder 22 of a user and are sufficient in size to cover the entire shoulder of the subject up to the neck 24. Because the heat pack 14 and pack cover 16 are thin and flexible, they easily wrap around the shoulder 22 of the patient. The heat pack 14 may be used on any number of different body parts effectively.

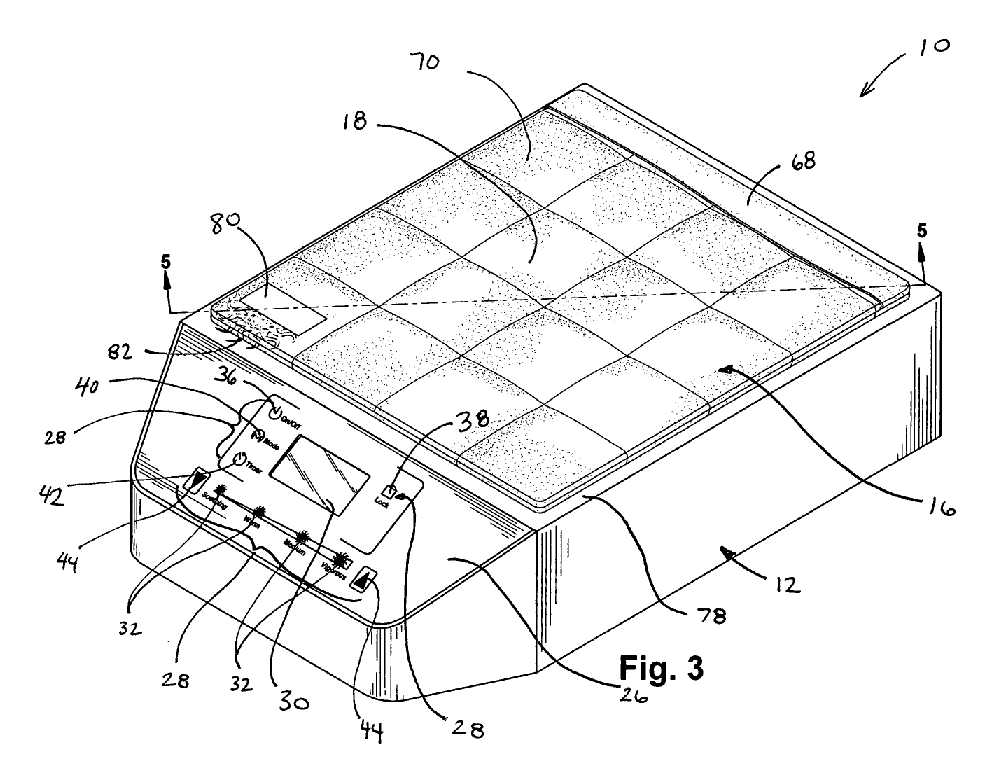

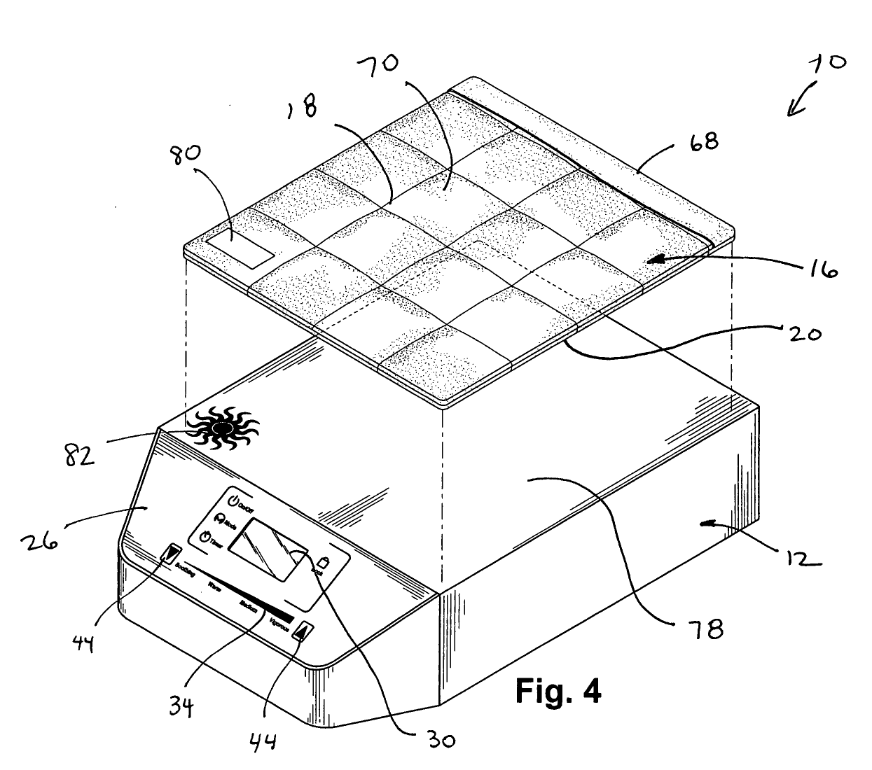

FIGS. 3 and 4 depict a heat pack 14 and cover 16 positioned on the induction charging unit 12. The induction charging unit 12 is utilized to heat the heat pack 14 to a prescribed temperature by energizing one of the layers of the heat pack via induction. The charging unit 12 has a face plate 26 with several inputs 28 and outputs 30. The primary output device 30 is an LCD screen. The inputs 28 are touch screen or mechanical buttons, or the like. The buttons may include underlying LEDs (not shown) for lighting the button under the face plate 26 so that a user can tell whether a button has been selected.

The charging unit 12 controls the temperature of the heat pack 14. A practitioner may enter the temperature manually, or select from a range of preset temperatures on the charging unit 12. As shown in FIG. 3, the user can select from four different temperature settings 32. FIG. 4 shows the induction charging unit 12 as having a sliding temperature scale 34 that will allow the user to select from among many different temperatures. A numeric temperature scale (not shown) may alternatively or also be positioned on the face plate 26 of the charging unit 12.

One charging unit 12 that may be utilized with this system is manufactured by CookTek Magna Wave Systems, of Chicago, Ill. under model Appogee MC 1800. This is a table top unit that works at 50 to 60 Hz using 1800 watts of power. The power source is a standard 120V wall outlet. This unit is equipped with Radio Frequency Identification (RFID) technology and Real Time Device (RTD) technology that enables the charging unit 12 to communication with the heat pack 14 and perform a series of commands. For instance, the charging unit 12 has an RFID antenna, RFID reader 86, and a processor for communicating with an RFID tag 52. Technology related to RFID and RTD operation is available from Therapy Solutions, Inc., of Wichita, Kans. An example charging unit 12 has a magnetic coil (not shown) that is sized based on the size of the heat pack 14. As an example, the magnetic coil could be 9.5 inches in diameter for a corresponding 12 in.×12 in. pack.

The charging unit 12 also includes software that allows for the setting of temperatures, times, power output, and temperature ranges. As previously discussed, external input and outputs on the device itself may include push buttons, switches, touch screens, one or more LCD displays, and graphics, among other input and output devices. The devices shown in FIGS. 3 and 4 include an on/off button or switch 36, a locking button or switch 38, a mode button or switch 40, a timer button or switch 42, and temperature selection buttons or switches 44 adjacent a temperature scale 46. Other types of input and output mechanisms may alternatively be used, including a touch screen or keyboard, among other known input and output devices.

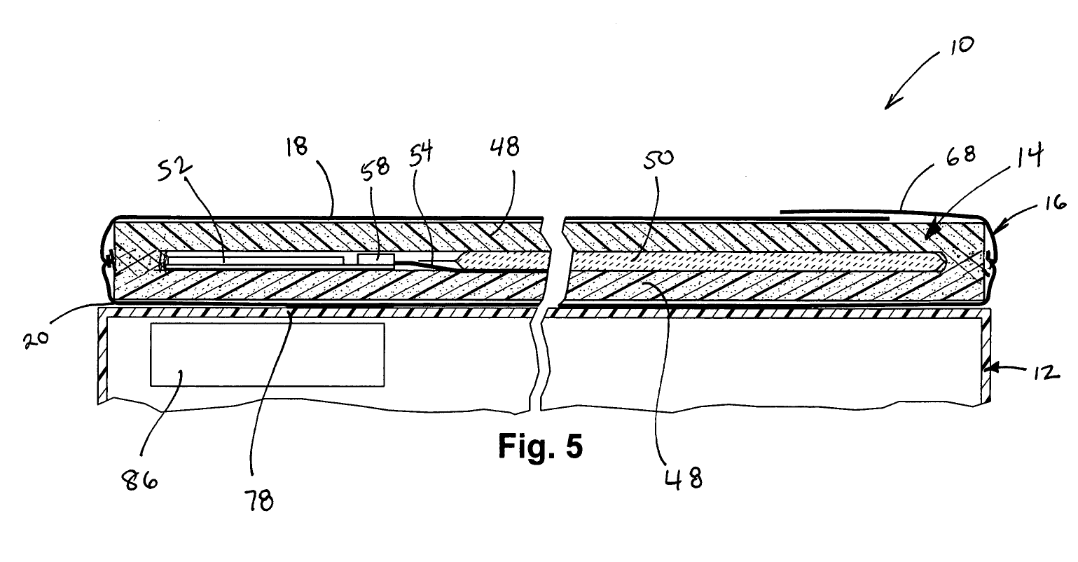

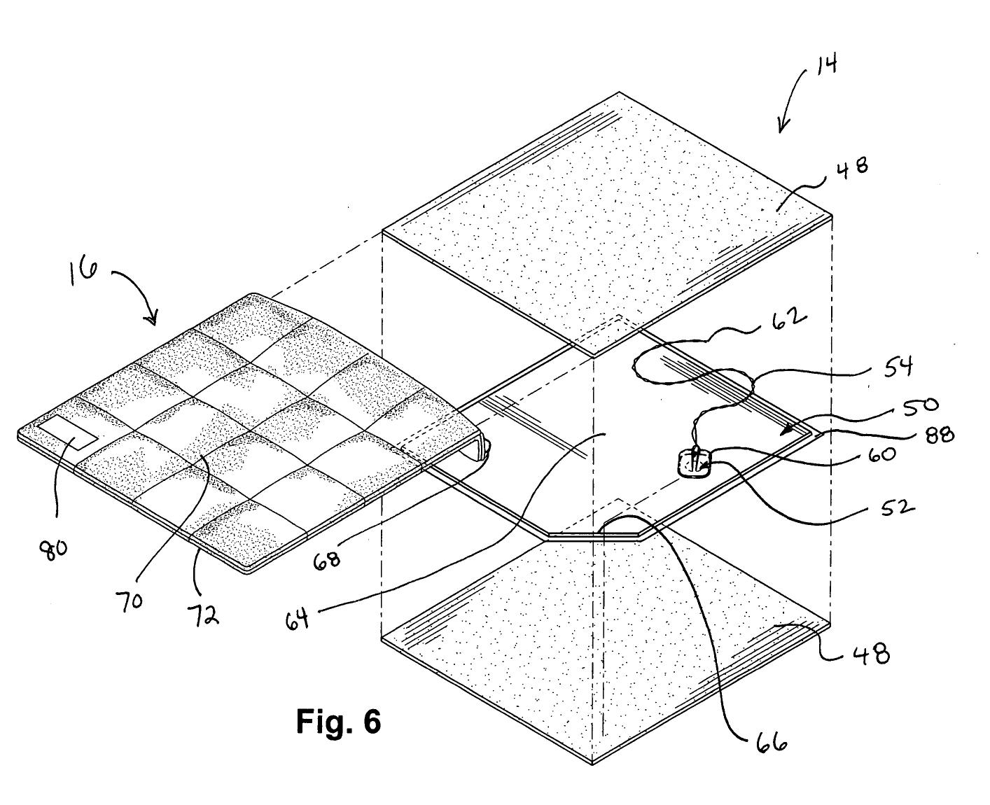

FIGS. 5 and 6 depict one configuration of the heat pack 14, positioned inside the pack cover 16. The heat pack 14 has been designed to exploit the most efficient means of heating based upon considerations of time, temperature saturation, and heat retention. The heat pack of this example includes three layers of material that are made of five base components. The base components include an elastomer, such as polyurethane; an energizing layer, such as laminated flexible graphite; a thermal conductivity enhancing element, such as powdered/flaked graphite; a phase change material or materials; and an RFID/RTD tag and sensor.

The heat pack 14 is constructed using two outer heat retaining elastomeric layers 48, with a layer of energizing material and the RFID/RTD tag and sensor sandwiched between the elastomeric layers. The energizing material may be flexible graphite which reacts well to magnetic energy produced by the charging unit 12. As such, the layer of graphite 50 serves as the heating element for the heat pack 14.

Graphite has proven to be an excellent source of heat when coupled with inductive energy. The graphite used may be a pressed sheet material that ranges in thickness from 0.005 inches to 0.3 inches. The size and shape of the graphite layer 50 is dictated by the size and shape of the antenna installed in the charging unit 12. Flexible graphite may be made very thin, such that is no more thick than a few sheets of paper. As a result, its light weight makes the heating element of the heat pack insignificant to the weight of the pack 14. Although graphite itself is brittle and limited to minimal stress, it may be laminated to thin polyurethane films 88 that encase the graphite. The polyurethane films 88 give the graphite greater flexibility and durability to contortion and stress. Graphite is also very cost effective.

The sheet of graphite 50 is approximately 1 inch smaller in scale than the outer shape of the elastomeric layers 48. For example, with a 12 inch×12 inch×0.5 inches thick heat pack 14, the graphite sheet 50 measures approximately 11 inches×11 inches. For each variation of pack size or shape, an example graphite sheet has a density of 70 pounds and a thickness of 0.015 inches.

The flexible graphite sheet 50 may be grafoil, a laminated graphite sheet, or other flexible graphite that measures between about 0.005 inches to 0.3 inches in thickness. Other thicknesses may be utilized. In addition, other materials may be utilized. For example, any type of energizing material may be used, including stainless steel. It is preferred that the material utilized be flexible in order for the heat pack 14 to conform to body parts of a patient.

One example of the elastomeric layers 48 of the heat pack 14 are constructed of a two part polyurethane gel material, such as that produced by Northstar Polymers of Minneapolis, Minn. as part number MPP-V37A. The two parts of the polyurethane gel material are identified as MPA-135 (part A) and PNA-157 (part B). MPA-135 is a prepolymer and PNA-157 is a curing agent. The materials are mixed at a ratio of about 1:2.2 (part A: part B) by weight or about 1:2.3 (part A: part B) by volume. The materials may be used in other ratios, ranging from about 1:2 to 1:3 by weight. More particularly, a preferred range is 1:2.1 to 1:2.9 by weight. The optimal mixture based upon volume is dictated based upon the size of the heat pack being poured. For example, a 12 inch×12 inch×0.5 inches thick heat pack is 72 ci or 1.49 quarts of the mixture. MPP-V37 has a durometer hardness of Shore OO 37 and a tensile strength of 85 psi. Product specifications for MPP-V37A and its components are available at www.tandemproducts.com/Northstar/MPP-V37A.htm, the disclosure of which is hereby incorporated by reference in its entirety. Other PU gels may be substituted, but it is preferred that they have a similar durometer and density characteristics, although this is not absolutely required.

A powdered/flaked raw graphite is added to the mixture and amounts to 20% by volume of the mixture in one example. The raw graphite powder is added to increase the thermal conductivity of the gel material, which, in turn, shortens heating time of the heat pack 14 and saturates the pack with more even heating. Another material may be substituted for the graphite flake, if desired, such as any thermally conductive material that can be ground down or flaked into small enough granules. Other forms of materials may also be added. Raw graphite may be supplied by EGC Enterprises, Inc. of Chardon, Ohio. Alternative examples use graphite power in a percentage by volume range of about 10% to about 30%.

A phase change material is also added to the mixture and amounts to 15% by volume of the mixture in this example. One phase change material that may be utilized is paraffin and silica based powder. These each have tremendous heat storage capabilities. The phase change material increases the heat retention of the polyurethane gel and provides increased time at therapeutic temperatures. Alternative examples use a phase change material in a percentage by volume of 10 to 30%. One phase change material that may be utilized with the mixture is a phase change powder supplied by Rubitherm GmbH of Kyritz, Germany under model number PX-52. PX-52 is a latent heat powder based on paraffins. The melting point is approximately 52° C., the average particle size is 250 μm, the specific heat capacity is 1.6 Kj/Kg, and the heat storage capacity is 103 Kj/Kg. Other information concerning the properties of this material may be found at www.rubitherm.com. Other phase change materials may also be used, such as sugars, waxes, synthetics, and the like. In another example, the combined powders of the graphite and phase change materials do not exceed 35% of the total volume of the mixture. Other examples include combinations of 20% graphite by volume and 10% phase change material by volume; 10% phase change material and 15% graphite by volume; and 10% graphite and 20% phase change material by volume.

The elastomeric layers 48 of the heat pack 14 may alternatively be made of a polyurethane, such as Sorbothane. Other types of materials that may be used are phase change materials (PCMs), such as waxes; polyurethane gels, such as two part polyurethane gels; polyurethane; polyethylenes, or urethane elastomers. Phase change materials may be those that transition at 250 degrees F, or at other temperatures. Example materials that may be utilized take a short amount of time to heat, such as under 5 minutes and preferably 2-3 minutes or less, and stay hot for at least 30 minutes. Other heat retentive materials may also be added to the materials discussed above to increase the temperature holding time for the heat pack 14. Thermal retentive materials may also be added to decrease the thickness of the heat pack 14 while providing the same length of time for heat retention. A polyurethane combined with a phase change material, such as a powder, can be utilized for the elastomeric layers 48.

Each heat pack 14 also may include an RFID tag 52 and an RTD 54 for measuring the temperature of the energizing layer 50 of the heat pack 14. The RFID tag 52 preferably includes an antenna 56 for communicating with an external reader 86 and/or writer in the charging unit 12. The RFID tag 52 and temperature sensor 56 are positioned inside the heat pack 14 such that the RFID tag 52 is hidden within the body of the pack 14. The RFID tag 52 is preferably sandwiched between the two elastomeric layers 48 and the RTD 54 is positioned adjacent the energizing layer 50 in order to accurately read the temperature of the energizing layer 50. The RFID tag 52 and RTD 54 may be spray or quick glued to the graphite, taped to the graphite, or may otherwise be put into contact with the graphite layer 50, such as trapped next to the graphite layer when the elastomeric layers 48 are poured.

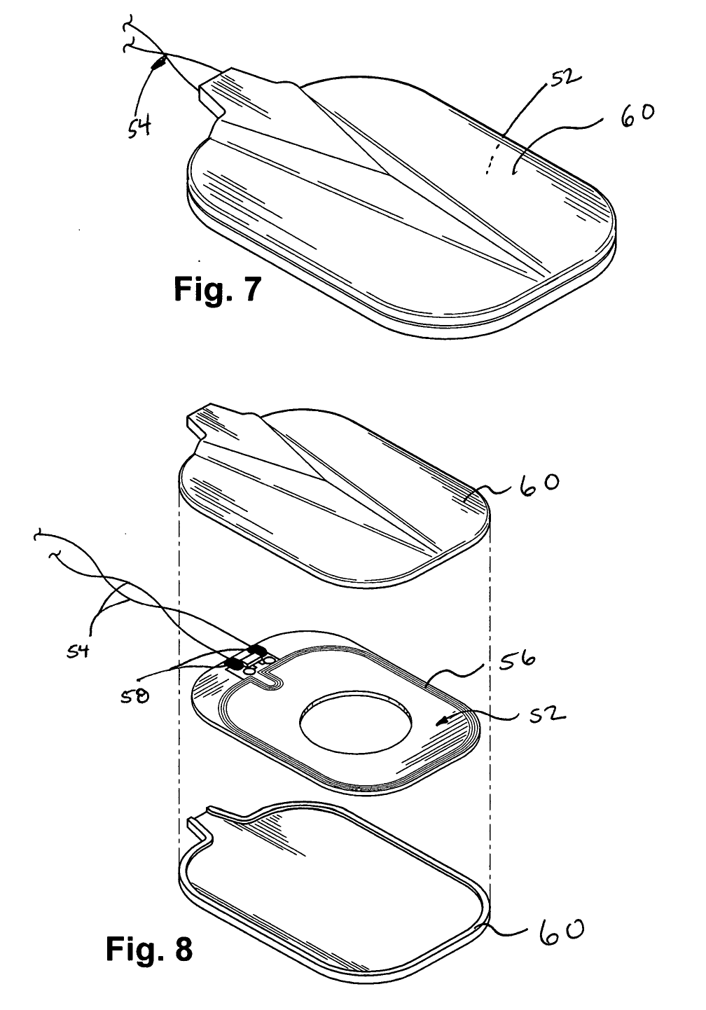

As shown in FIGS. 7 and 8, the RTDs 54 are soldered 58 to the RFID tag 52 and the tag 52 is encased in a protective casing or coating 60, such as a protective polyurethane shell. The protective casing 60 is used to protect the RFID 52 from damage, or from disconnecting the RTDs 54 from the RFID tag 52. The protective shell 60 helps the RFID tag 52 to withstand the flexibility that the heat pack 14 will endure. The RTDs 54 are preferably positioned with their end 62 in a center area 64 of the heat pack 14, for more efficient temperature readings. One type of RFID 52 tag that may be utilized with the examples is supplied by Tagsys RFID, of Huveaune, France.

While the examples depicted herein utilized RFID/RTD technology for temperature purposes, the heat pack 14 could be utilized without RFID or RTD technology. The heat pack 14 could alternatively be heated by simply dialing in a temperature on an induction heating device 12, without the need for RFID technology.

Each heating element 50 is laminated between the two sheets of thermo set polyurethane 48 and may be vacuum heat sealed, although this is not absolutely required. The overall laminate is then cut to 0.5 inches less than the finished size of the pack cover 16. One corner 66 of the heating element is trimmed and sealed to allow an opening for an RFID tag 52. The heating element 50 is trimmed in order to minimize interference between the graphite 50 and the RFID signal to the charging unit 12.

Because of the type of materials utilized in the above-described example, the assembled heat pack 14 may be tacky to the touch. Talcum, cornstarch, or other powder may be applied to the surface of the heat pack 14 in order to remove the tackiness.

The heat pack can be any number of thicknesses, depending upon the thickness of the various layers and the number of layers. For example, the elastomeric layers could be ¼ inch thick, ⅛ inch thick, 1/10th inch thick, or ½ inch thick, among other thicknesses. The heat pack could have a total of 3 to 20 layers, with the elastomeric 48 and graphite 50 layers being stacked upon one another in sandwich-like style. The elastomeric layers 48 could be different within the sandwich. For example, even in a three layer sandwich, the outer elastomeric layers could be different materials from one another. The heat pack 14 demonstrates good multidirectional flexibility and durability.

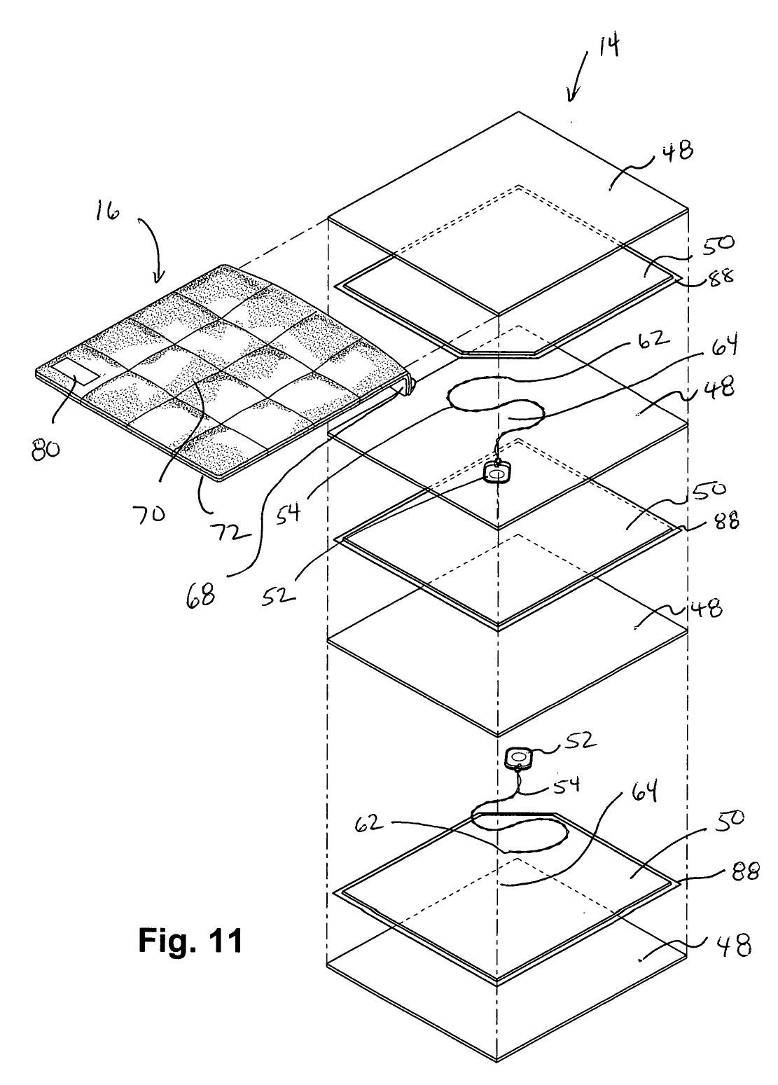

As shown in FIGS. 10 and 11, the heat pack may comprise further layers than those discussed above. For example, three graphite layers 50 may be used with four elastomeric layers 48, or more. When multiple heating elements 50 are used, multiple RFID/RTDs 52/54 may also be utilized, although a single RFID/RTD may be used. As shown, the RFID tags 52 may be positioned at opposite sides of the heat pack 14.

The system 10 includes a pack cover 16 that is utilized to cover the heat pack 14. The cover 16 may simply be a sack that has two like layers, one positioned on the top of the heat pack and another positioned on the bottom the heat pack. The cover 16 can include an overlapping flap 68, such as that shown. Alternatively, the cover 16 can be closed by other means, such as zippers, hook and loop tape, buttons, or otherwise (not shown). If desired, the cover 16 may include features designed to improve the performance of the heat pack 14. In particular, the cover 16 may be designed for directional use. The cover 16 is custom fit for each individual shape and size of heat pack 14.

In one example, the cover 16 utilizes a combination of thermal retentive material 70, a breathable surface 72, and a moisture/cleanliness barrier 74. The breathable surface 72 is a thin, single layer of material, such as a cotton/synthetic blend, that is designed to allow heat to transfer easily from the heat pack 14 to a subjects skin. This surface is intended to be used as the bottom 20 of the pack cover 16. The top 18 of the pack cover 16, that portion that faces outwardly, is designed to capture the heat of the pack and minimize heat loss to the open air. The top 18 of the cover 16 includes a thermal insulating material 70 that is positioned between two layers, such as layers of synthetic blends. One example of an insulating material is Insul-Bright, produced by The Warm Company of Seattle, Wash. The insulating material may be quilted between the two layers, such as shown in FIG. 1. An alternative insulating layer may be neoprene, either alone or together with surrounding layers. The use of different bottom and top layers for the pack cover 16 helps to maximize the heat transfer to the subject body part while insulating the heat pack 14 from room temperature. Other types of materials may also be used for the layers of the cover.

In addition, the pack cover 16 may include a moisture/cleanliness barrier 74, as shown in FIG. 9. The barrier is a thin layer of moisture absorbing material that is attached to the bottom 20 of the cover 16 via hook and loop tape 76, or by any other removable means of attachment. This barrier layer 74 serves at least two functions. First, the barrier 74 may be moistened such that moisture is applied along with heat. Second, the barrier 74 offers a clean surface that can be easily removed and replaced with a new cloth for each new patient usage. This reduces any risk of cross-contamination between subjects. The barrier 74 may be washed after every use, while the pack cover 16 can continually be used throughout the day. This not only minimizes laundry, since the entire pack cover 16 does not need to be washed after each patient, but also reduces prep time since the heat pack 14 doesn't need to be removed and replaced with a new pack cover 16. The barrier 74 and pack cover 16 are both preferably machine washable. The pack cover 16 and barrier 74 layers are preferably made of a material that has good durability. Examples of possible materials for any of the layers include terry cloth, micro fleece, nylon, spandex, neoprene, or other materials.

In use, the system 10 is designed to make the use of superficial heat easier, faster, cleaner, and more efficient. The charging unit 12 is space saving, easily disinfected, and runs off a common 120V wall outlet. Once the charging unit 12 is plugged in, it is ready for operation. The charging unit 12 may have a “stand by” mode, when not in use for a period of time, in order to conserve energy.

Operation of the system is relatively simple. The heat pack 14 is positioned on a top surface 78 of an induction charging unit 12 and is turned on such that magnetic energy is communicated to the energizing layer 50 of the heat pack 14. The changing magnetic field of the charging unit 12 induces electric currents in the energizing layer 50, which results in heating of the energizing layer 50. Upon heating of the graphite layer 50, heat is transferred to the elastomeric layers 48.

Prior to heating of the heat pack 14, the heat pack is positioned inside the pack cover 16. The pack cover 16 includes an indicator 80 that is positioned on the top exterior surface 18 of the pack cover 16. The indicator 80 signals where the RFID tag 52 is located in the heat pack 14. The indicator 80 may be a sewn on tag, a surface treatment to the exterior layer of material of the pack cover 16, or a marking of any type. The charging unit 12 has a corresponding locator 82 in one corner of the unit, that signifies the location for placing the indicator 80 of the pack cover 16. An example of this is shown in FIG. 4. The induction charging unit 12 may have a limited proximity range in order to limit unwanted heating of heat packs 14 in the vicinity of the charging unit 12. For example, the charging unit 12 may have a range of 4 inches, such that the RFID 52 tag of the heat pack 14 must be within 4 inches of the top surface 78 of the induction charging unit 12. When the pack cover 16 is positioned properly on the charging unit 12, the indicator 80 of the pack cover 16 will be positioned over the locator 82 on the charging unit 12.

Once the heat pack 14 and pack cover 16 are positioned on the charging unit 12, the practitioner turns the unit on by touching the on/off button 36, at which point the unit will display a set temperature and an actual temperature on the LCD screen 30. The unit will proceed to automatically read the RFID tag 52 in the heat pack 14 and display the current temperature reading in an “actual temp” location of the LCD 30. The practitioner may then choose one of four possible heat settings, such as Soothing, Warm, Medium, or Vigorous heat, and the LCD 30 will depict the corresponding temperature on the LCD as the “set temp”. The charging unit 12 then energizes the energizing layer 50 of the heat pack 14. As the heat pack 14 climbs in temperature, the “actual temp” displayed on the LCD 30 shows the current temperature until the temperature reaches the “set temp”. Once the “set temp” is reached, the charging unit 12 holds that temperature until the heat pack 14 is removed from the unit 12. The practitioner may then apply the barrier 74, which may be moistened, if desired. The barrier 74 may alternatively be positioned on the bottom 20 of the cover 16 prior to heating. The heat pack 14 is then ready for use on a subject.

Due to the nature of the human body and physiology, each person reacts to heat therapy differently. Some patients react differently to dry and moist heat. Some patients perceive temperatures to be higher than other patients. For this reason, four different preset temperature settings are used in the example shown in FIG. 3. Research indicates that at 114 degrees, human tissue can be damaged. As a result, the Vigorous setting is designed to provide a target 112 degree F skin temperature. Because this may not be suitable for all patients, the other settings are present to accommodate variations from person to person. The Vigorous temperature setting heats the graphite layer to 165 degrees F, the Medium temperature setting heats the graphite layer to 155 degrees F, the Warm setting heats the graphite layer to 145 degrees F, and the Soothing setting heats the graphite layer to 135 degrees F.

As an alternative to the above, the charging unit may be programmed such that a practitioner can simply input a desired temperature. With this example, the “set temp” would correspond to the temperature input by the practitioner. For this purpose, a number keypad may be provided on the charging unit (not shown). A practitioner may select a “set temp” based on a predetermined desired temperature where the temperature scale is a sliding scale, such as that shown in FIG. 4. In FIG. 4, the practitioner gradually moves up the temperature scale by pressing the buttons adjacent the scale until the “set temp” desired is reached. A slide bar input device could be used instead of the depicted buttons 44.

As discussed above, the RTD 54 is preferably positioned in proximity to the graphite layer 50 so that it can accurately read the core temperature of the heat pack 14. The RFID tag 52 communicates with the RTD 54 to continually monitor the temperature of the pack 14. The induction charging unit 12 also utilizes an RFID reader and antenna (not shown) for communicating with the RFID tag on the heat pack 14. When the heat pack 14 is positioned on the induction charging unit 12, the RFID reader 86 communicates with the RFID tag 52 to determine such things as proximity, any information that is written into the RFID tag 52, and temperature. The RFID reader 86 is coupled to a microprocessor and can continually communicate with the RFID tag 52 to monitor and adjust the temperature of the heat pack 14 when the heat pack 14 is in proximity to the charging unit 12. In the examples shown, the charging unit 12 includes inputs for selecting a temperature for the heat pack 14. In another example, a separate card (not shown) can be utilized and scanned into the induction charging unit RFID reader to program the heating instructions for each heat pack 14. Other techniques and devices are also envisioned for input and output to the reader 86 and processor. Other materials may also be utilized.

An example method of making a heat pack is also provided. In the method, the polyurethane (PU) is first measured and poured from its two part mixture. Because the PU is rationed by weight, the mixture is poured into containers over a scale. For mixing purposes, part B is poured first and measured, then part A is added and measured. A paddle mixer is used to evenly mix the PU. As the mixture becomes homogenous, the 20% of graphite powder and 15% of phase change powder is slowly added to the mixture, until an even, smooth mixture is achieved. Other percentages and ratios may alternatively be utilized, as discussed above.

After creation of the mixture, the second step involves pouring half of the mixture into a mold that is configured in the shape desired for the heat pack 14. Once the first layer is poured, it is required to set for a period of time. For example, it may set for up to an hour before further processing is performed.

The graphite sheet 50 is then prepared. The RFID 52 is aligned with a dissected corner 66 of the graphite sheet 50, which is laminated between two thin film layers 88, and the RTD leads 54 are then positioned in a serpentine S-like pattern until the tip 62 of the RTD 54 is aligned over the center 64 of the graphite sheet 50. This serpentine helps to eliminate stress and strain on the RTD lead 54 and its attachment to the RFID tag 52 when the heat pack 14 is flexed. The RFID 52 and RTD 54 are then affixed to the surface of the graphite layer 50. They may be affixed using high temperature tape or other affixing means (not shown).

After the graphite sheet 50 is prepared, the graphite sheet 50 with electronics attached is then firmly positioned on to the center of the first half of the pack. Then the second half of the mixture is poured over the top of the graphite layer 50, electronics and first layer. This results in bonding of the first and second layers 48 and encapsulating of the electronics 52, 54 and graphite layer 50. The dissected corner 60 of the heat pack 14 is then marked to identify the location of the RFID tag 52. FIG. 5 illustrate how the elastomeric layers join together to encapsulate the graphite. FIG. 10 shows the layers 48 being separated from one another by a space. This is only for illustration purposes and it should be understood that adjoining elastomer layers 48 will join together around the perimeter of the heat pack 14.

The material can be placed in an oven to decrease the set time. However, the PU will set at room temperature in 5-7 hours. After the PU has set, the heat pack 14 can be removed from the mold and doused with cornstarch or other powder to remove any tackiness of the materials and to provide a smooth touch.

While graphite has been described as the primary material for the energizing layer 50 of the heat pack, any type of distinct sheet material that possesses the high temperature and chemical resistance characters of graphite, as well as additional characteristics of flexibility and resilience, may be utilized. In addition, while the examples are described in the context of heating pads, the example configurations described herein could be used in other therapeutic heating or non-therapeutic heating. The heat pack materials could be used, for example, to line a piece of clothing in order to keep a person warm under extreme cold conditions. Other examples of use are also anticipated.

While the previously described examples involve a practitioner inputting a desired temperature to the charging unit, the RFID tag of each heat pack may alternatively be programmed with a prescribed temperature. In this case, when the heat pack is positioned on a charging unit, the reader of the charging unit reads the prescribed temperature from the RFID tag and automatically heats the heat pack to the prescribed temperature, without requiring input from a practitioner. In this embodiment, multiple heat level heat packs are provided, with each having different temperature settings programmed into the RFID. In order to assist practitioners in using these heat packs, the heat packs or pack covers may be color coded. These heat packs may help practitioners to avoid input errors.

In addition to induction heating, another type of heating could be microwave heating, where the heat pack is placed into a microwave and heated for a specified period of time.

The word “substantially,” if used herein, is a term of estimation.

While various features of the claimed invention are presented above, it should be understood that the features might be used singly or in any combination thereof. Therefore, the claimed invention is not to be limited to only the specific examples depicted herein.

Further, it should be understood that variations and modifications may occur to those skilled in the art to which the claimed invention pertains. The examples described herein are exemplary of the claimed invention. The disclosure may enable those skilled in the art to make and use examples having alternative elements that likewise correspond to the elements of the invention recited in the claims. The intended scope of the invention may thus include other examples that do not differ or that insubstantially differ from the literal language of the claims. The scope of the present invention is accordingly defined as set forth in the appended claims.

1. A heat pack for therapeutic use comprising:
a first layer of an elastomeric heat retaining material;
a second layer of an elastomeric heat retaining material; and
a first flexible sheet of an energizing material positioned between the first and second layers of elastomeric heat retaining materials, said energizing material being conducive to inductive heating.

2. The heat pack of claim 1, wherein the first and second layers are made of a material selected from one or more materials from the group of polyurethanes, gel polyurethanes, waxes, and urethane elastomers

3. The heat pack of claim 1, wherein the first flexible sheet is a graphite material.

4. The heat pack of claim 1, wherein the first and second layers comprise a polyurethane elastomer, a heat retentive material additive, and a phase change material.

5. The heat pack of claim 3, wherein the first flexible graphite sheet is grafoil.

6. The heat pack of claim 1, wherein an RFID tag is associated with the first flexible energizing sheet.

7. The heat pack of claim 6, wherein the RFID tag is covered with a protective coating to deter damage of the RFID during use of the heat pack.

8. The heat pack of claim 6, wherein the RFID tag is coupled to an RTD temperature sensor, and both the RFID tag and the RTD temperature sensor are coupled to the first flexible energizing sheet.

9. The heat pack of claim 8, wherein the RFID tag is positioned in the vicinity of one corner of the pack and an end of the RTD temperature sensor is positioned in the vicinity of the center of the pack, and the pack is rectangular in shape.

10. The heat pack of claim 6, wherein the energizing sheet has a dissected corner area, and the RFID tag is positioned in the dissected corner area.

11. The heat pack of claim 1, further comprising a pack cover for covering and enclosing the first layer, the second layer, and the first energizing sheet.

12. The heat pack of claim 11, wherein the pack cover is made of a cloth material.

13. The heat pack of claim 11, wherein the pack cover comprises a top layer and a bottom layer, with the top layer being an insulating layer, and the bottom layer being a breathable layer, and further comprising a barrier layer that is removably coupled to the breathable layer, wherein the barrier layer is positionable against the skin of a subject.

14. The heat pack of claim 11, wherein the barrier layer is made of a moisture absorbing material.

15. The heat pack of claim 3, further comprising more than two layers of elastomeric material and more than one layer of flexible graphite, with the layers of flexible graphite being positioned between the layers of elastomeric material, wherein an RFID tag is associated with at least one of the flexible graphite layers.

16. A system for providing superficial heat to a subject in a therapeutic setting comprising:
an induction charging unit; and
the heat pack of claim 1.

17. The system of claim 16, further comprising:
an RFID tag coupled to the heat pack; and
an RFID reader and RFID antenna coupled to the induction charging unit, wherein the RFID tag is in communication with the RFID reader via the RFID antenna when the heat pack is positioned in proximity to the induction charging unit.

18. The system of claim 17, further comprising an indicator positioned on the heat pack to identify the location of the RFID tag and a locator positioned on a surface of the induction charging unit indicating a location of the RFID reader, wherein in use, the indicator of the heat pack is positioned on top of the locator on the charging unit in order to allow for effective communication between the RFID tag and the RFID reader.

19. The system of claim 16, further comprising:
an RFID tag coupled to a temperature sensor, said tag and temperature sensor being coupled to the energizing sheet of the heat pack;
an RFID reader associated with the induction charging unit, wherein the RFID tag communicates temperature information to the RFID reader in order to heat the flexible energizing layer to a prescribed temperature.

20. The system of claim 19, wherein the induction charging unit includes a microprocessor having programming for accepting a prescribed temperature based upon an input from a user, and the microprocessor is programmed to heat the energizing layer to the prescribed temperature based upon input from the temperature sensor and RFID tag to the RFID reader of the charging unit.

21. The system of claim 17, wherein a prescribed temperature is stored in the RFID tag and the RFID reader is capable of reading the prescribed temperature from the RFID tag, and a microprocessor having programming is coupled to the charging unit such that when the microprocessor of the charging unit receives the prescribed temperature from the RFID tag of a heat pack, the charging unit heats the energizing layer to the prescribed temperature.

22. The system of claim 17, wherein the RFID reader has a proximity range such that the reader can only read the RFID tag of the heat pack when the RFID tag is in close proximity to the induction charging unit.

23. The system of claim 17, wherein the induction charging unit includes a microprocessor and a mechanism for inputting a prescribed temperature to the microprocessor, and the RFID tag is configured to communicate an actual temperature reading of the energizing layer to the microprocessor such that the charging unit heats the energizing layer of the heat pack such that the actual temperature meets the prescribed temperature.

24. A chemical composition for an elastomeric heat retentive material comprising:
a polyurethane gel material;
about 10 to 30% by volume graphite; and
about 10 to 30% by volume phase change material, wherein the combined amount of graphite and phase change material does not exceed about 35% of the total volume of the mixture.

25. The chemical composition of claim 24, wherein the polyurethane material is a two part polyurethane comprising a prepolymer and curing agent in a weight ratio ranging from about 1:2 to about 1:3 of prepolymer to curing agent.

26. The chemical composition of claim 25, wherein the phase change material is paraffin and silica based powder, the graphite is a powder, and the two part polyurethane gel material has a durometer hardness of about Shore OO 37 and a tensile strength of about 65 psi.

27. The chemical composition of claim 25, wherein the two part polyurethane gel material ratio is about 1:2.2 by weight.

28. The chemical composition of claim 25, wherein the two part polyurethane gel material ratio is about 1:2.1 to about 1:2.9 by weight.

29. The chemical composition of claim 25, wherein the graphite has a volume percentage of about 20% of the total mixture, the phase change material has a volume percentage of about 15% of the total mixture, and the weight ratio of the two part polyurethane gel materials is about 1:2.2 of prepolymer to curing agent.

30. A method of manufacturing a heat pack comprising:
mixing a mixture of materials to product a heat retentive elastomeric material;
pouring at least part of the mixture into a mold to produce a first layer of elastomeric material;
positioning an energizing material over the first layer of elastomeric material;
pouring at least part of the remaining mixture over the first layer of elastomeric material and the energizing material to produce a second layer of elastomeric material and to trap the energizing material between the first and second layers; and
removing the layered pack from the mold.

31. The method of claim 30, further comprising:
prior to pouring the second layer of elastomeric material, positioning an RFID tag in a dissected corner of the energizing material and positioning an end of an RTD lead that is coupled to the RFID tag in a central area of the energizing material such that the RTD lead is one of touching, or in close proximity to the energizing material.

32. The method of claim 30, further comprising waiting until the layered pack has cured before removing the layered pack from the mold; and dousing the layered pack with a powder-like material to remove any tackiness.

33. The method of claim 30, further comprising, covering the layered pack with a cloth-like enclosure.
